(19) United States
(12) Patent Application Publication
(10) Pub. No.: US 2025/0213172 A1
(43) Pub. Date: Jul. 3, 2025

(54) SYSTEM AND METHODS FOR AUDITORY STIMULATION TO AFFECT SLEEP

(71) Applicants: NeuroAcoustics, Inc., Atlanta, GA (US); Instituto Tecnológico de Buenos Aires, Autónoma de Buenos Aires (AR)

(72) Inventors: Rodrigo Ezequiel Ramele, Buenos Aires (AR); Cecilia Forcato, Buenos Aires (AR)

(21) Appl. No.: 18/979,743

(22) Filed: Dec. 13, 2024

Related U.S. Application Data
(60) Provisional application No. 63/617,178, filed on Jan. 3, 2024.

(51) Int. Cl. A61B 5/374; A61F 11/00; A61N 1/36

(52) U.S. Cl. CPC A61B 5/374; A61F 11/00; A61N 1/36

(57) ABSTRACT

A system and method of auditory stimulation to affect sleep of a subject utilize monitored brain wave activity signals while the subject is asleep to detect an indication of the start of a slow oscillation using a set of detection parameters. Then, a time delay to apply before auditory stimulation is determined, auditory stimulation to affect the slow oscillation is applied after the time delay, and a reward value for the auditory stimulation is calculated by evaluating the brain wave activity signal after applying the auditory stimulation. The length of the time delay to be applied prior to subsequent applications of the auditory stimulation associated with subsequent slow oscillations is adjusted based on the reward value to provide personalized and adaptive auditory stimulation. Additionally, the system and method can use the monitored brain wave activity signals to generate subject-specific detection parameters for adaptive detection of slow oscillations.

### CROSS-REFERENCE TO RELATED APPLICATION

This application claims priority to U.S. Provisional Patent Application No. 63/617,178, filed Jan. 3, 2024, entitled “SYSTEM AND METHODS FOR AUDITORY STIMULATION TO AFFECT SLEEP,” which is expressly incorporated herein by reference in its entirety.

### BACKGROUND

Quality sleep is essential for both physical and mental health. In humans, sleep can be generally divided into two main sleep stages: rapid eye movement (REM) sleep and non-REM sleep, which typically alternate in a cyclic manner. Non-REM sleep plays several fundamental biological functions including, but not limited to, physical restoration and repairing processes, cognitive improvement, immune system strengthening, clearance of aberrant proteins in the brain such as tau and beta-amyloid (a precursor to Alzheimer disease), axon sprouting, mood regulation, and overall well-being promotion. Poor quality sleep, e.g., due to sleep deprivation or receiving less than the recommended hours of sleep, can therefore have several detrimental effects at a physical and mental level. For example, poor quality sleep hampers the repair of skeletal muscles after exercise (e.g., as non-REM sleep regulates the secretion of testosterone, cortisol, and growth hormone). Moreover, poor quality sleep can affect the replenishment of muscular glycogen, hindering recovery and diminishing subsequent performance in activities, and impairing cognition as well as mood.

Non-REM sleep can be further divided into light sleep (i.e., Stages 1 and 2) and slow wave sleep (i.e., Stages 3 and 4) or “SWS.” SWS is characterized by high-amplitude cortical slow oscillations (herein, “SO,” e.g., around 0.8 Hz), thalamo-cortical spindles (e.g., 10-15 Hz), and hippocampal ripples (e.g., around 80 Hz in humans). Slow oscillation activity is often much more subdued in light sleep (e.g., Stage 2) compared to SWS. Slow oscillations are related to the maintenance of synaptic homeostasis in the brain. That is, the equilibrium at the neuronal connection strength. During wakefulness, as a result of the continuous encoding of information, synaptic strength increases in the brain. This leads to an elevation in cellular energy requirements and a reduction in the signal-to noise ratio.

In non-pathological brains, the amplitude of slow oscillations peaks during the first hours of sleep and then decreases. This dissipation is essential for the brain—on the one hand, it prevents an oversaturation of synaptic strength allowing us to encode new information when awake (e.g., promoting learning) and, on the other hand, it improves mood. Furthermore, non-REM slow oscillations are responsible for the consolidation of recently acquired memories. During non-REM sleep new memories are reactivated in the hippocampus, transferred, and redistributed into the neocortex favoring long-term memory consolidation. This hippocampal-neocortical “dialog” underlying memory consolidation is served by the temporal coupling between hippocampal ripples, thalamocortical spindles and neocortical slow oscillations.

In normal aging, as well as in various pathologies, slow oscillations can be reduced in quantity as well as in amplitude, causing a direct impact to cognition, mood, and other aspects of wellbeing. Additionally, the oscillatory behavior of SO is affected due to aging. While in younger population SO are characterized in trains as oscillatory activity, elder population exhibits greater number of isolated SO (separated more than 3 seconds from any other SO). For example, non-REM sleep decreases with normal aging such that a reduction of the quality (e.g., number and amplitude) of slow oscillations is already observed at the age of 40, impairing the ability to learn and consolidate new information. Many pathologies also affect slow oscillation quality. In temporal lobe epilepsy, for example, there is a temporal desynchronization between ripples and spindles during non-REM sleep that explains the impairment in cognition in these patients. Patients suffering from depressive disorder have a dysregulation of the sleep cycle. In these patients, not only is the time spent in non-REM sleep decreased, but the distribution of slow oscillations is atypical. More specifically, the amplitude of slow oscillations is typically lower than in healthy people and no dissipation of these oscillations is observed during a night of sleep. This dysregulation is directly associated with the mood disturbances of these patients.

In yet another example, patients that suffered acquired brain injuries, such as stroke or traumatic brain injury (TBI), often show changes in the sleep architecture that affects rehabilitation. Slow oscillations promote local axonal sprouting, favoring rehabilitation after stroke. Young healthy people that sleep fewer hours than the recommended amount, such as Olympic athletes and elite sportspeople, often show a subpar quality of sleep compared to non-athletes. This negatively impacts all aspects of their athletic performance, spanning from training to in-game execution and recovery. Prioritizing adequate and quality sleep is paramount for a healthy and competitive life to optimize physical recovery, mental health, performance, cognition as well as rehabilitation.

### SUMMARY

One implementation of the present disclosure is a method of auditory stimulation to affect sleep of a subject, the method including: monitoring a brain wave activity signal while the subject is asleep; detecting, from the brain wave activity signal, an indication of the start of a slow oscillation using a set of detection parameters generated for the subject, the set of detection parameters based on characteristics of the slow oscillation prior to hyperpolarization; determining a time delay to apply before auditory stimulation responsive to detecting the start of the slow oscillation, wherein the time delay is applied after detecting a trigger point in an upstate of the slow oscillation or after depolarization; applying auditory stimulation to affect the slow oscillation after the time delay, the auditory stimulation emitted by an audio output device; calculating a reward value for the auditory stimulation by evaluating the brain wave activity signal after applying the auditory stimulation, the reward value calculated in part by parsing the brain wave activity signal; and adjusting, based on the reward value, a length of the time delay to be applied prior to subsequent applications of the auditory stimulation associated with subsequent slow oscillations.

Another implementation of the present disclosure is a system for auditory stimulation to affect sleep, the system including: a first sensor configured to record brain wave activity in a subject; an audio output device; one or more processors; and memory having instructions stored thereon that, when executed by the one or more processors, cause the system to: monitor brain wave activity signals while the subject is asleep using the first sensor; detect, from the brain wave activity signal, an indication of the start of a slow oscillation using a set of detection parameters generated for the subject, the set of detection parameters based on characteristics of the slow oscillation prior to hyperpolarization; determine a time delay to apply before auditory stimulation responsive to detecting the start of the slow oscillation, wherein the time delay is applied after detecting a trigger point in an upstate of the slow oscillation or after depolarization; emitting, by the audio output device, auditory stimulation to affect the slow oscillation after the time delay; calculate a reward value for the auditory stimulation by evaluating the brain wave activity signal after applying the auditory stimulation, the reward value calculated in part by parsing the brain wave activity signal; and adjust, based on the reward value, a length of the time delay to be applied prior to subsequent applications of the auditory stimulation associated with subsequent slow oscillations.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a diagram of an example processing pipeline for closed-loop auditory stimulation (CLAS) to affect sleep, according to some implementations.

FIG. 2 is a block diagram of a CLAS system, according to some implementations.

FIG. 3 is a block diagram of the interaction between components of the CLAS system of FIG. 2, according to some implementations.

FIGS. 4A and 4B are diagrams of example sensor montages positioned on a subject, according to some implementations.

FIG. 6 is a flow chart of a process for generating subject-specific slow oscillation detection parameters, according to some implementations.

FIG. 7 is a flow chart of a process for adaptively detecting slow oscillations and applying auditory stimulation, according to some implementations.

FIG. 5 is a graph of the components of an example slow oscillation, according to some implementations.

FIG. 8 is a graph of an example slow oscillation with and without an applied auditory stimulus, according to some implementations.

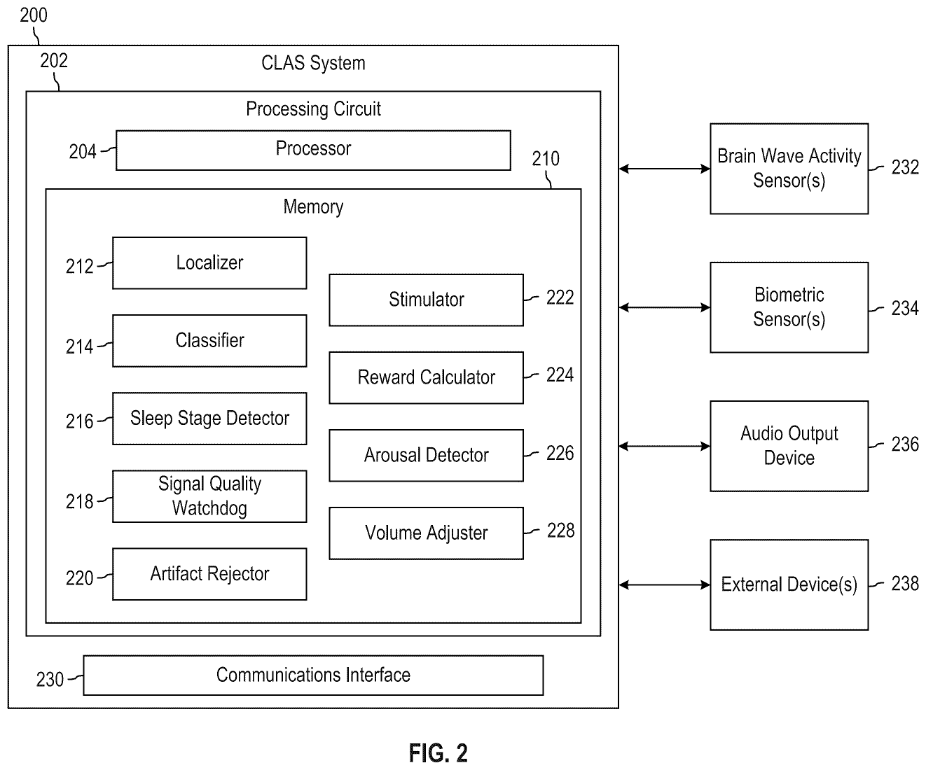

FIG. 9 is a block diagram of the interaction between components of the CLAS system of FIG. 2 for developing a training data set, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a system and methods for closed-loop auditory stimulation (CLAS) to affect sleep are shown, according to various implementations. CLAS refers to the use of auditory stimulus (e.g., specific tones) at low intensity, such as pink noise, to synchronize neuronal cortical activity during sleep, thereby increasing the quality (e.g., size and number) of slow oscillations. Generally, the technique employed by the disclosed system and methods begins with the detection of the negative peak of an endogenous slow oscillation and synchronous emission of a tone (e.g., a pink noise burst). After a time delay, at a point where another slow oscillation would naturally appear, another tone can be emitted to enhance subsequent slow oscillations. This enhancement of slow oscillations during sleep can have many notable impacts in a variety of subjects, such as improved cognition (e.g., memory and attention) in healthy and/or cognitively impaired adults, reduced symptomatology of depression, increased sports performance, improved rehabilitation of acquired brain injuries (e.g., stroke, TBI, etc.), improving cognition in epileptic patients, and more. Additionally, as discussed in greater detail below, the system and methods described herein can be used to promote the appearance of isolated slow oscillations by performing exogenous auditory stimulations while a subject is in non-REM sleep stages.

Notably, the above-mentioned technique for CLAS implemented by the disclosed system and methods differs from other CLAS techniques by being adaptable to subjects of different demographics and/or having different pathologies. For example, sleep in older adults is often characterized by the presence of isolated and/or transient slow oscillation events that cannot be precisely characterized in an oscillatory manner. In this regard, existing CLAS techniques—which typically assume that slow oscillation events are periodic or oscillatory—are typically not suitable for treating older adults. More generally, existing CLAS techniques are often limited in their application to, at most, one demographic, and can still provide lackluster results due to the sometime unpredictable nature of slow oscillations. It is additionally worth noting that existing CLAS techniques have, so far, been limited mainly to laboratory or academic use due to the cost, size, and complexity of equipment.

In order to effectively apply synchronized auditory closed loop stimulation, a more precise data-driven approach is desirable to adaptively identify and then characterize slow oscillation events. To effectively localize and identify these components, any real-time system must deal with the problem of recognizing slow oscillation events before they can be fully observed to apply auditory stimulation at the right time. The disclosed system and methods address these points, and various limitations of current CLAS techniques as discussed above, two-fold: through the generation of subject-specific SO detection parameters for the fast and accurate detection of slow oscillation events, and through adaptive stimulation timing that considers how previous auditory stimulations have impacted subsequent slow oscillations. These and other features of the disclosed system and methods are described in greater detail below.

### Overview

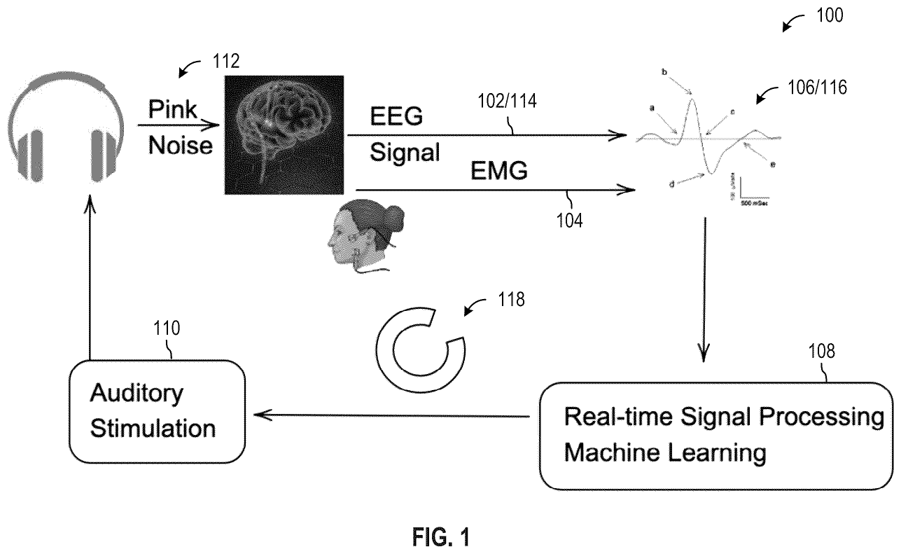

Turning first to FIG. 1, a diagram of an example processing pipeline 100 for CLAS to affect sleep is shown, according to some implementations. Specifically, in some respects, pipeline 100 represents an overview of the CLAS technique introduced above and implemented by the disclosed system and methods, which are described in greater detail below. Pipeline 100 begins with the recordation of brain wave activity signals (102) and, in some cases, biometric signals (104) while a subject is sleeping. In the example shown, the brain wave activity signals are electroencephalogram (EEG) signals, e.g., captured by a sensor array position on a subject's head as they sleep. The biometric signals are shown as electromyograph (EMG) signals, e.g., captured by the sensor array position on the subject's head or by another sensor array, but can also include other biometric signals such as electrooculograph (EOG) signals. Additional discussion of the specific brain wave activity and biometric signals recorded, and the corresponding sensors for capturing said recordings, is provided below with respect to FIG. 2.

From the brain wave activity signals, a candidate signal indicative of a slow oscillation is detected (106). In particular, the recorded brain wave activity signals are continuously analyzed against “detection parameters” and, if the detection parameters are met, a “candidate signal” event is triggered. The detection parameters can include, for example, a peak-to-peak amplitude and a negative threshold; however, other detection parameters are discussed below. In this regard, the brain wave activity signal may be evaluated to detect a negative and sharp downward deflection of the signal and that the raw value of the signal falls below a negative threshold. This technique, e.g., of continuously analyzing brain wave activity signals against parameterized thresholds, results in the quick and efficient detection of suspected slow oscillations as they are appearing (e.g., as opposed to detection after the slow oscillation is fully formed). Further, the detection parameters can be customized to the subject from which the brain wave activity signals are being recorded for even faster and more accurate detection.

To this point, pipeline 100 can further include a processing step (108) for generating and/or adjusting the detection parameters to be subject-specific. To generate subject-specific detection parameters, a plurality of candidate signals may first be detected using initial detection parameters and recorded. A trained classifier, which is a type of machine learning model, is then used to classify each of the plurality of candidate signals as either “indicative of a slow oscillation” or “not indicative of a slow oscillation.” These steps of detecting and classifying the candidate signals can be repeated a number of times, each time with a different set of detection parameters, to generate two sample data sets of candidate signals—one set that are indicative of a slow oscillation and one set that are not indicative of a slow oscillation. Then, the initial (or finally utilized) detection parameters can be adjusted based on the sample data sets to generate subject-specific detection parameters.

After an initial calibration, e.g., to generate the subject-specific detection parameters, or after selecting a previously generated set of subject-specific detection parameters, the subject-specific detection parameters are utilized for subsequent real-time detection of slow oscillations. When a candidate signal indicative of a slow oscillation is detected, a stimulation event is triggered (110). During a stimulation event, auditory stimulation in the form of a pink noise burst is emitted (112), e.g., via an audio output device. The audio output device may be a headset or a pair of headphones, ear buds, a speaker or set of speakers, or the like. However, prior to emitting the auditory stimulation, a time delay is implemented to synchronize the auditory stimulation with the slow oscillation. In other words, the stimulation event includes waiting a certain number of time steps after detection of the slow oscillation before emitting auditory stimulation to ensure that the auditory stimulation is applied at an optimal moment in the slow oscillation.

Throughout the application of the auditory stimulation, the brain wave activity signals are monitored (114) and the resulting slow oscillation signal is parsed into its components—particularly, to identify a first zero-crossing after detection of the slow oscillation and a second zero-crossing after the auditory stimulation. Based on these zero-crossing points, a second valley in the signal (e.g., shown in FIG. 5) is identified and used to calculate a “reward” for the auditory stimulation (116). The “reward” can be considered, in some respects, as a value indicative of the improvement of the slow oscillation due to the auditory stimulation. In turn, the reward can be used for reinforcement learning (118) to modify the time delay between detection of subsequently slow oscillations and corresponding application of auditory stimulation. In other words, the timing of subsequent pink noise bursts can be adjusted to adapt the stimulation to the specific subject being evaluated/treated, thereby synchronizing the auditory stimulation with the slow oscillation.

Throughout the process illustrated by pipeline 100, the aforementioned biometric signals can also be monitored to determine the subject's sleep stage (e.g., sometimes referred to as “sleep scoring”), such that stimulation is only applied when the subject is in non-REM, Stage 2 or a deeper sleep (e.g., Stages 3 or 4). If the subject is determined to have exited Stage 2 sleep and entered Stage 1, for example, the brain wave activity monitoring and/or auditory stimulation may be disengaged. In some implementations, EMG signals (e.g., captured from sensors on or around the subject's head/face) are used to detect arousal of the subject and/or to determine REM stage. In some implementations, EOG signals are used to detect REM and/or differentiate REM versus non-REM. A more detailed discussion of the disclosed system and methods for implementing pipeline 100 is provided below.

### Adaptive Auditory Stimulation System

Referring now to FIG. 2, a block diagram of a CLAS system 200 for affecting sleep is shown, according to some implementations. As mentioned above, CLAS system 200 is generally configured to implement the CLAS process illustrated by pipeline 100 which, notably, is adaptive across subjects of different demographic and/or having different pathologies. For example, unlike existing CLAS techniques, CLAS system 200 implements a technique that can be used to treat both young patients, which may have more regular and/or periodic SOs, and older patients, which tend to have sporadic (e.g., transient) SOs that cannot be accurately detected in real-time using current techniques. Additionally, CLAS system 200 enables the adaptive adjustment of auditory stimulation, e.g., by monitoring the effects of auditory stimulation on resulting slow oscillation activity, to improve treatment. These and other notably features of CLAS system 200 will become clearer with the following description.

CLAS system 200 is shown to include a processing circuit 202 that includes a processor 204 and a memory 210. Processor 204 can be a general-purpose processor, an application specific integrated circuit (ASIC), one or more field programmable gate arrays (FPGAs), a group of processing components (e.g., a central processing unit (CPU)), or other suitable electronic processing structures. In some implementations, processor 204 is configured to execute program code stored on memory 210 to cause CLAS system 200 to perform one or more operations, as described below in greater detail. In some implementations, CLAS system 200 can be part of another computing device (e.g., a smartphone, a laptop, etc.) such that the components of CLAS system 200 may be shared with, or the same as, the host device. For example, if CLAS system 200 is implemented via a smartphone, then CLAS system 200 may utilize the processing circuit, processor(s), and/or memory of the smartphone to perform the functions described herein. Building further on this example, in some implementations, certain functions of CLAS system 200 may be conducted on a remote device, e.g., to alleviate some of the computation requirement on CLAS system 200.

Memory 210 can include one or more devices (e.g., memory units, memory devices, storage devices, etc.) for storing data and/or computer code for completing and/or facilitating the various processes described in the present disclosure. In some implementations, memory 210 includes tangible (e.g., non-transitory), computer-readable media that stores code or instructions executable by processor 204. Tangible, computer-readable media refers to any physical media that is capable of providing data that causes CLAS system 200 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 210 can include random access memory (RAM), read-only memory (ROM), erasable programmable read-only memory (EPROM), electronically erasable programmable read-only memory (EEPROM), hard drive storage, temporary storage, non-volatile memory, flash memory, optical memory, or any other suitable memory for storing software objects and/or computer instructions. Memory 210 can include database components, object code components, script components, or any other type of information structure for supporting the various activities and information structures described in the present disclosure. Memory 210 can be communicably connected to processor 204, such as via processing circuit 202, and can include computer code for executing (e.g., by processor 204) one or more processes described herein.

While shown as individual components, it will be appreciated that processor 204 and/or memory 210 can be implemented using a variety of different types and quantities of processors and memory. For example, processor 204 may represent a single processing device or multiple processing devices. Similarly, memory 210 may represent a single memory device or multiple memory devices. Additionally, in some implementations, CLAS system 200 may be implemented within a single computing device (e.g., one controller, one housing, etc.). In other implementations, CLAS system 200 may be distributed across multiple servers or computers (e.g., that can exist in distributed locations). For example, CLAS system 200 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

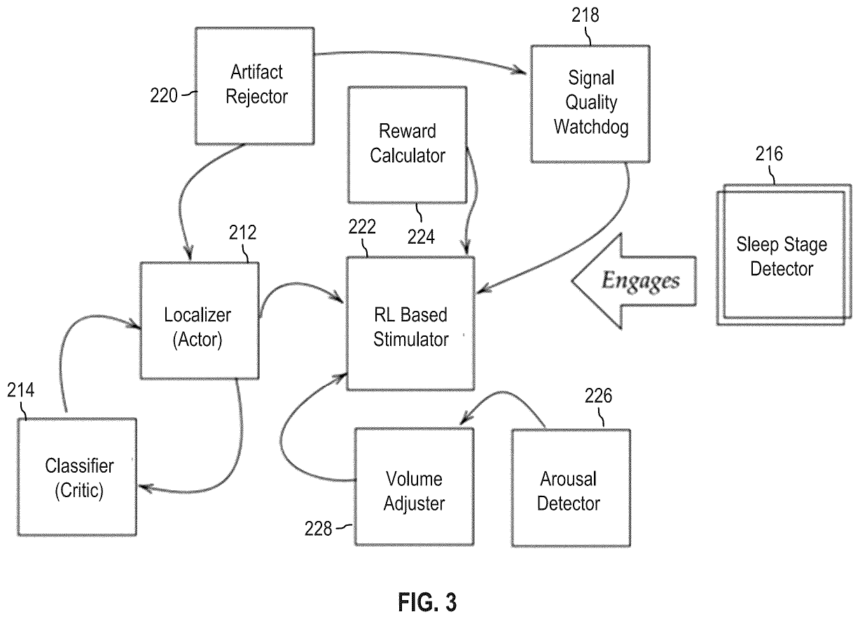

Memory 210 is shown to include a localizer 212 configured to detect slow oscillation events. More particularly, localizer 212 evaluates brain wave activity signals to identify when a slow oscillation is appearing, e.g., by detecting hyperpolarization. The “brain wave activity signals” monitored by localizer 212 generally refer to signals received from brain wave activity sensor(s) 232, which record electrical activity of the brain. To this point, brain wave activity sensor(s) 232 can include a plurality of electrodes for generating EEG recordings. Localizer 212 is generally configured to analyze one EEG single channel at a time; however, it should be appreciated that multiple EEG signal channels can be recorded simultaneously and/or the specific channel being analyzed by localizer 212 may be selectable. Additionally, or alternatively, in some implementations, a combination of EEG channels can be used, e.g., using spatial filtering techniques. Additional description of brain wave activity sensor(s) 232 is provided below with respect to FIG. 3.

In detecting slow oscillations, localizer 212 is generally configured to compare the recorded brain wave activity signals (e.g., a single-channel EEG signal) against a set of detection parameters. Specifically, localizer 212 may continuously analyze segments of the received brain wave activity signal to determine if the set of detection parameters are met and, if so, can flag a portion of the brain wave activity signal a “candidate signal” indicative of a slow oscillation. In some implementations, the brain wave activity signal is analyzed in one second segments; however, the present disclosure is not intended to be limiting in this regard. The specific detection parameters utilized to detect slow oscillations are generally based on unique characteristics of slow oscillations and can vary between types of slow oscillations, subject demographics, and more. For example, different detection parameters can be used to detect certain types of slow oscillations (e.g., type I, type II, etc.) and the detection parameters generally vary between subjects. Regardless, the detection parameters are generally based on distinct features of slow oscillations before the “upstate” (e.g., as shown in FIG. 5).

In some implementations, the set of detection parameters utilized by localizer 212 includes a peak-to-peak amplitude threshold and a negative threshold, but more, fewer, or different parameters may be used. In some such implementations, localizer 212 aims to detect a negative and sharp downward deflection of the brain wave activity signal. Thus, a portion of the brain wave activity signal is identified as a candidate signal if there is a sharp difference between a positive and negative peak (e.g., if the peak-to-peak amplitude threshold is met) and the raw value of the signal goes below the negative threshold. Other example detection parameters can include, but are not limited to, a time delay between a point where the negative threshold is reached and the maximum point from the peak-to-peak amplitude, a location of the negative peak, a location of a negative-to-positive zero-crossing, a location of a positive peak, an amplitude of the positive peak before downward deflection, a frequency phase and so on.

In some implementations, localizer 212 can also be configured to detect different slow oscillations components which are characterized by different combinations of parameters. Put another way, localizer 212 can be configured to detect different sizes and/or types of slow oscillations, which may appear differently and have different characteristics. For example, not all slow oscillations are identical; some may have different peak or peak-to-peak amplitudes, different frequency components, etc. “High” and “low” slow oscillations, for example, could be differentiated by the presence or absence of certain nested frequency bands, which may have different functional characteristics. Thus, the set of detection parameters utilized by localizer 212 can vary based on the characteristics of the slow oscillation(s) to be detected. In some implementations, localizer 212 can be configured to utilize multiple different sets of detection parameters, e.g., each associated with a different “type” of slow oscillation. For example, localizer 212 may generate (and subsequently use, for detection) a first set of detection parameters for detecting slow oscillations that are classified as ‘type I’ or ‘type II’ according to morphological features based on the parsing procedure of the slow oscillation. In this regard, localizer 212 is not limited in the number of different sets of detection parameters, or type of detection parameters, that can be used to differentiate and/or detect different types of slow oscillations.

In some implementations, when a candidate signal is identified (e.g., when a candidate signal event is triggered), localizer 212 is configured to capture a segment of the brain wave activity signal, e.g., having a fixed length. More generally, localizer 212 can trigger subsequent processing steps (e.g., discussed below) when a slow oscillation is detected. In some implementations, localizer 212 additionally considers a refractory period as part of the candidate signal detection process. The refractory period refers to a minimum amount of time (e.g., a time delay) between detection of candidate signals. In other words, after identifying a first candidate signal, localizer 212 waits a set amount of time defined by the refractory period before identifying a second candidate signal. This helps to ensure that candidate signals are indicative of different slow oscillations. In some implementations, the refractory period is adjustable and/or is modified during calibration. The refractory period may also be different when stimulation is forced (e.g., in implementations where CLAS system 200 is used to cause or “kickstart” slow oscillations).

As mentioned above, one notable feature of CLAS system 200 is the functionality to generate detection parameters (e.g., for detecting candidate signals indicative of slow oscillations) that are subject-specific, resulting in quicker and more accurate slow oscillation detection. For example, slow oscillation characteristics and timing can vary greatly between subjects of different demographics and/or having different pathologies; thus, adapting the detection parameters to an individual subject can help to ensure that the slow oscillations associated with the individual subject are accurately identified. To generate subject-specific detection parameters, localizer 212 and a classifier 214 (also of memory 210, as shown) may cooperatively perform a detection parameter calibration process, e.g., during a calibration phase prior to auditory stimulation. In this regard, localizer 212 may be referred to as an “actor” and classifier 214 as a “critic” of localizer 212. To better understand the interactions between localizer 212 and classifier 214, as well as other components of memory 210 as described below, additional reference may be made herein to FIG. 3.

Classifier 214 is generally configured to determine a probability that a candidate signal segment recorded by localizer 212 is associated with a slow oscillation, and therefore is indicative of the start of a slow oscillation. In this regard, a primary function of classifier 214 is to determine a class/label for candidate signals identified by localizer 212. The class/label may be a determination that the candidate signals “is indicative of a slow oscillation” or “is not indicative of a slow oscillation.” Put another way, the class/label determined by classifier 214 may be a binary indication (e.g., yes/no, 0/1, etc.) of whether a candidate signal is, or is not, associated with a slow oscillation. Classifier 214 therefore may include a classifier, which is a type of machine learning model that predicts a class/label for a given input (e.g., in this case, a segment of the brain wave activity signal). In some implementations, classifier 214 can additionally output a confidence score in each class prediction which can be used to filter out results with low confidence.

Classifier 214 can generally be, or include, any suitable type of machine learning model for classifying signal segments (e.g., captured by localizer 212), such as a linear discriminant analysis (LDA) model, a support vector machine (SVM), or a multi-layer perceptron; however, other suitable types of machine-learning based classifiers are contemplated herein. In some implementations, classifier 214 is a unary classifier. A unary classifier, as known to those of skill in the art, is a type of one-class or binary classifier. As an example, a unary classifier may output a “yes” or “no” to answer the questions, “is this detected candidate signal indicative of a slow oscillation?” In other implementations, classifier 214 is a multiclass classifier that is capable of differentiating between different types of slow oscillations. In this regard, classifier 214 can provide the ground-truth between more than two options, e.g., type I, type II, or false slow oscillations. For example, classifier 214 can be trained (e.g., as described below) to differentiate between different predefined types of slow oscillations, e.g., based on their associated characteristics. It should also be noted that, in some implementations, the information received by classifier 214 (e.g., signal segments captured by localizer 212), may be further processed using a spatial filtering algorithm, such as Laplacian smoothing, independent component analysis (ICA), or common spatial patterns (CSP).

In some implementations, classifier 214 is trained using a semi-supervised training process; however, unsupervised and supervised training processes are also contemplated herein. Semi-supervised training utilizes datasets of example brain wave activity signal segments, e.g., captured by CLAS system 200, that have been annotated by sleep research experts. In one example, experts may review example brain wave activity signal segments and mark slow oscillations to generate a training set. Specifically, experts can mark the start and/or end of slow oscillations, and/or may add labels identifying different types of slow oscillations. In some cases, the experts may also mark signal segments that were wrongfully identified as slow oscillations and/or may change labels for slow oscillations that were wrongfully identified as being of a specific type. To this point, in some implementations, classifier 214 may initially be trained using generic and/or previously captured brain wave activity signal data not associated with a subject that will be using CLAS system 200. Then, CLAS system 200 may be used to generate a sample dataset of brain wave activity signals from the subject for additional training, where candidate signals captured during generation of the sample dataset are manually annotated by experts, to be used to retrain classifier 214 before full deployment of CLAS system 200. Additional discussion of the training process for classifier 214 is described below with respect to FIG. 9.

Returning to the discussion of subject-specific detection parameters, one role of classifier 214 is to provide a ground-truth for determining whether a candidate signal is, in fact, indicative of a slow oscillation. During a calibration phase, e.g., prior to stimulation, localizer 212 is configured to randomly select an initial set of detection parameters (e.g., (t₁,t₂)¹), e.g., around a standardized set of values. Then, localizer 212 utilizes the initial set of detection parameters (e.g., (t₁,t₂)¹) to identify a candidate signal associated with a possible slow oscillation. A segment of the candidate signal is provided to classifier 214 (see FIG. 3) which, in turn, determines a ground-truth label for the candidate signal (e.g., “indicative of a slow oscillation” or “not indicative of a slow oscillation”). Localizer 212 then randomly selects a second set of detection parameters (e.g., (t₁,t₂)²), uses the second set of detection parameters to identify a second candidate signal, and again classifier 214 determines a ground-truth label. This process can be repeated several times to generate two sample datasets from both classes—one of signal segments (e.g., candidate signals) that are determined to be associated with slow oscillations and another of signal segments that are determined to be false positives. Alternatively, or additionally, in implementations where localizer 212 is configured to detect and/or identify different types of slow oscillations, multiple sample datasets may be generated—one for each type or “class” of slow oscillation.

Using these sample datasets, the subject-specific detection parameters (e.g., (t₁,t₂)*) can be generated. In some implementations, the subject-specific detection parameters (e.g., (t₁,t₂)*) are generated adjusting each detect parameter value (e.g., peak-to-peak amplitude, negative threshold, etc.) using a linear thresholding algorithm that allows for some form of segmentation. Examples of suitable linear thresholding algorithms are linear perceptrons, SVMs, or the Otsu method; however, other suitable techniques are contemplated herein. In any case, the linear thresholding algorithm is used to find the separation hyperplane (e.g., in two dimensions for two free parameters) for estimating parameters. In some implementations, the subject-specific detection parameters (e.g., (t₁,t₂)*) are generated by adjusting one of a standardized set of detection parameters (e.g., used to randomly select the initial detection parameters as mentioned above), the initial set of detection parameters (e.g., (t₁,t₂)¹), or the last-used set of detection parameters (e.g., (t₁,t₂)²). With the subject-specific detection parameters generated, localizer 212 can be used to detect slow oscillations during a stimulation phase (e.g., when using CLAS system 200 for treatment).

It is worth noting that localizer 212 and classifier 214 may cooperatively operate in an online or offline manner, e.g., to generate subject-specific detection parameters. In online operations, subject-specific detection parameters are generated during an initial calibration phase using live data collected from a subject. For example, the calibration phase may be implemented during the first 20 minutes or one hour that the subject is asleep, or until the subject-specific detection parameters meet or exceed and threshold (e.g., an error rate threshold). In offline operations, data may be collected by CLAS system 200 (e.g., or another system that can collect brain wave activity signals) over a period of time before generating the subject-specific detection parameters. For example, the subject may be monitored overnight such that a night's worth of brain wave activity signals are collected. Then, the subject-specific detection parameters can be generated in a similar manner to that described above. In a similar manner, subject-specific detection parameters could be generated for different sleep cycles or stages.

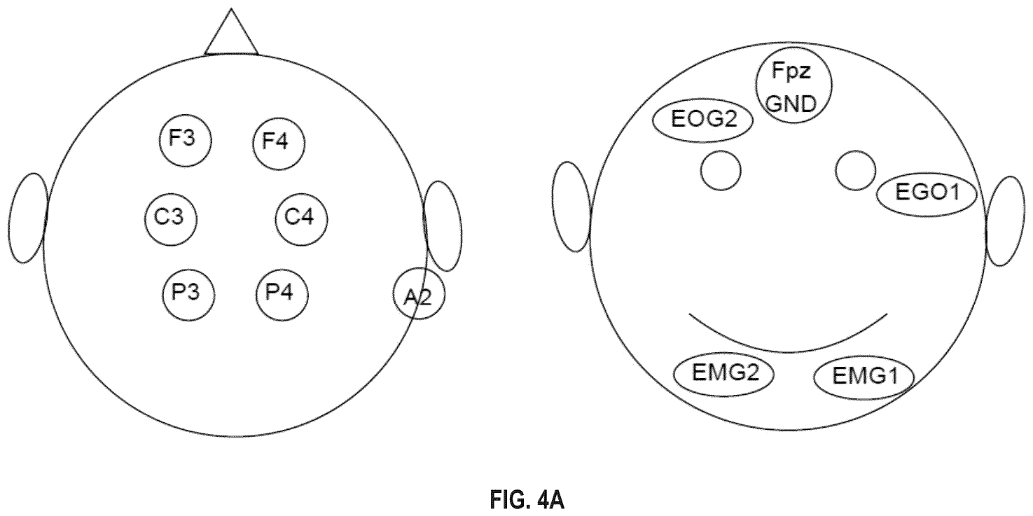

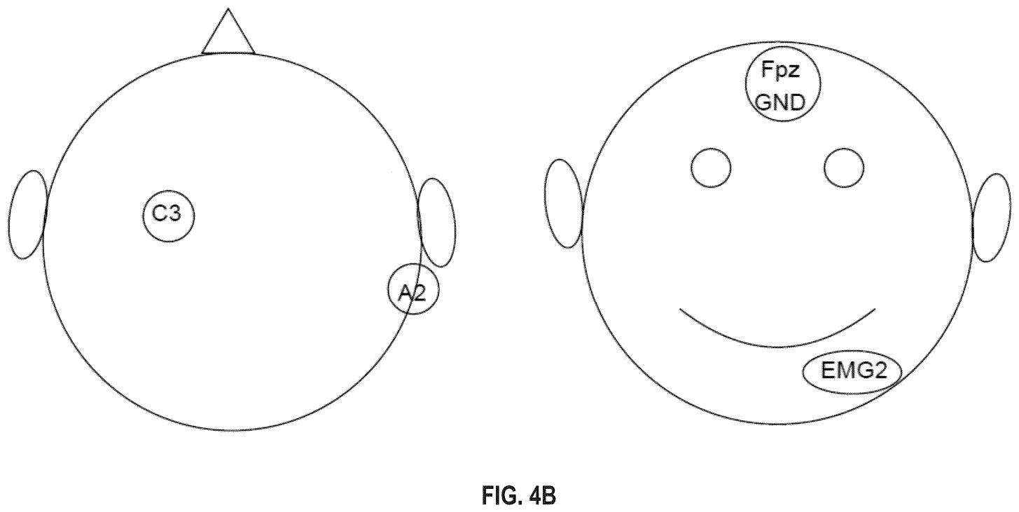

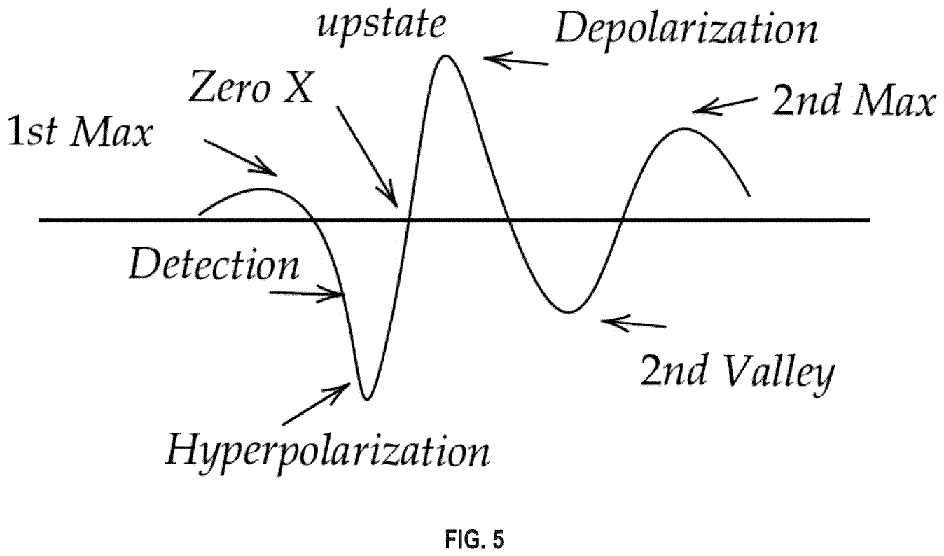

As shown, memory 210 further includes a sleep stage detector 216 configured to engage/disengage a stimulation phase implemented by CLAS system 200. Generally, applying auditory stimulation at the proper sleep stage (e.g., Stage 2 or deeper) is important; otherwise, the auditory stimulation may be ineffective and/or may cause the subject to wake up. Thus, sleep stage detector 216 is configured to enable and disable slow oscillation detection and/or auditory stimulation based on a subject's sleep stage at any given time. For example, if sleep stage detector 216 determines that the subject is in Stage 1, the process of detecting slow oscillations and/or applying auditory stimulation (e.g., pipeline 100) may be disabled. Sleep stage detector 216 generally obtains, as inputs, signals from brain wave activity sensor(s) 232 and/or biometric sensor(s) 234 and, based on an evaluation of the sensor data, enables or disables operations of one of more other components of memory 210 (see FIG. 3). In some implementations, sleep stage detector 216 controls other components of memory 210 directly or sleep stage detector 216 outputs a sleep stage value (e.g., “Stage 2,” “REM,” etc.) that can be referenced by the other components of memory 210. As described in greater detail with respect to FIG. 4, below, biometric sensor(s) 234 generally include one or more sensors for measuring EMG and EOG signals which can be used to detect muscle activity (e.g., of muscle in the subject's face) and to differentiate REM versus non-REM, respectively.

In some implementations, sleep stage detector 216 is configured to trigger/initiate a timer that, when expired, indicates that the subject is in non-REM, Stage 2 sleep. In turn, determining that the subject is in at least Stage 2 sleep, or deeper, can be used to initiate localizer 212, e.g., to being monitoring for potential slow oscillations. In some such implementations, sleep stage detector 216 calculates an average standard deviation of an EMG signal (e.g., received from at least one of biometric sensor(s) 234) and subsequently monitors the EMG signal to determine if the EMG signal exceeds three standard deviations above the average standard deviation (referred to herein as a “movement threshold”). In some implementations, sleep stage detector 216 continuously or periodically checks the EMG signal against the average standard deviation. In some implementations, sleep stage detector 216 utilizes a sliding window to calculate the average standard deviation of the EMG signal. If the EMG signal is determined not to exceed three standard deviations, sleep stage detector 216 searches for the first k-complex (e.g., a signal component similar to the slow oscillation) and triggers/initiates a timer. The timer may be 10 minutes, in some implementations, but the present disclosure is not limiting in this regard. Once the timer expires, it is assumed that the subject is in non-REM, Stage 2 sleep. In conjunction, sleep stage detector 216 may monitor an EOG signal (e.g., received from at least one of biometric sensor(s) 234) and, each time the EOG signal is determined to exceed a threshold value (e.g., a peak above/below 75 uV) and a standard deviation of the EMG signal is below the movement threshold, sleep stage detector 216 concludes that the subject is in REM sleep. This “REM” output may be held for a length of time (e.g., one minute), after which sleep stage detector 216 reverts back to an indication of “non-REM” or “wakefulness.”

Memory 210 is further shown to include a signal quality watchdog 218 configured to detect if a given brain wave activity signal is acceptable for further processing and/or slow oscillation detection and, if not, select another sensor channel and/or engage/disengage the stimulation process. Specifically, signal quality watchdog 218 monitors the brain wave activity signals to determine whether the signal(s) exceed a first threshold. In addition, signal quality watchdog 218 can also check if a standard deviation of the signal(s) falls below a second threshold. In some implementations, the first threshold is 400 uV, and the second threshold is 5 uV; however, the present disclosure is not intended to be limiting in this regard. If a brain wave activity signal meets one or both of these conditions, signal quality watchdog 218 can evaluate additional available channels to select one that has a valid signal for a period (e.g., one minute). To this point, the functionality of signal quality watchdog 218 is mainly focused on determining the quality of brain wave activity signals, e.g., in terms of the received sensor data.

In contrast, memory 210 can also include an artifact rejector 220 that looks mainly for movements, snoring, or other events that may invalidate a brain wave activity signal. In this regard, artifact rejector 220 is generally configured to identify signals from any of brain wave activity sensor(s) 232, and also biometric sensor(s) 234, that may be artifacts (e.g., noise) and, therefore, that are not suitable for further processing. In some implementations, memory 210 is engaged after a slow oscillation is detected but prior to auditory stimulation. Generally, artifact rejector 220 compares the brain wave activity and biometric signals received from brain wave activity sensor(s) 232 and biometric sensor(s) 234, respectively, to predefined thresholds and, if the threshold condition is met, rejects the corresponding signal components as artifacts. With respect to brain wave activity signals, artifact rejector 220 may determine whether the signal (e.g., the EEG signal) exceeds a threshold of, e.g., 200 uV±. If not, artifact rejector 220 may flag at least the corresponding segment of the signal as an artifact. In conjunction, auditory stimulation and/or any further analysis are suspended until a refractory period (e.g., one minute) has passed. Similarly, artifact rejector 220 can reject biometric signals if they meet certain criteria.

Stimulator 222 is configured to determine parameters for auditory stimulation (e.g., timing) and to cause the emission of auditory stimulation via an audio output device 236. Generally, stimulator 222 is engaged by localizer 212 responsive to the detection of a slow oscillation (see also FIG. 3). That is, responsive to detecting the start of a slow oscillation (e.g., using subject-specific detection variables), localizer 212 may provide an indication to stimulator 222 or may otherwise engage stimulator 222, causing stimulator 222 to initiate a stimulation action event. Stimulator 222 then continuously analyzes the brain wave activity signal (e.g., at each of a plurality of successive time steps) to identify a point for triggering stimulation. In some implementations, this “stimulation trigger” point is when the signal changes from a negative value to a positive value, indicating that the slow oscillation is most likely in the upstate (e.g., as illustrated in FIG. 5, described below). For example, some research has found that improve or “optimal” enhancement of slow oscillations can be achieved if the stimulation is performed in the upstate. However, the flexibility of stimulator 222—and, moreover, CLAS system 200—allows for the “stimulation trigger” point and/or timing of actual stimulation to be adjusted. For example, in some cases, it may be desirable to apply auditory stimulation in the down state of the slow oscillation (e.g., as shown in FIG. 5), in which case stimulator 222 can be easily adapted to adjust stimulation timing as discussed below.

Once the “stimulation trigger” point is detected (e.g., a negative-to-positive shift of the brain wave activity), stimulator 222 selects an action to perform. The “action,” in this case, is a time delay until auditory stimulation is triggered. In some implementations, the time delay is defined by a number of time steps to wait until the auditory stimulation is triggered after detection of the upstate. In some such implementations, the length of each time step correlates to a sampling frequency of brain wave activity sensor(s) 232. Stimulator 222 may initiate a timer or counter based on the selected time delay and, when the timer or counter expires or reaches a set time/count, auditory stimulation is triggered, in which case stimulator 222 causes audio output device 236 to output the auditory stimulation.

As mentioned above, the auditory stimulation is typically a prescribed tone, such as a pink noise burst. Specifically, in some implementations, the auditory stimulation is a pink noise burst lasting 200 milliseconds. Accordingly, audio output device 236 can include any device that is capable of producing and emitting sound. For example, audio output device 236 may be a headset (e.g., a pair of headphones) or a speaker positioned next to, or near, the subject; however, the present disclosure is not limiting in this regard. It should also be appreciated that, in some implementations, stimulator 222 is further configured to trigger the application of vibrotactile stimulation (e.g., in conjunction with the auditory stimulation). Vibrotactile stimulation generally refers to the “feeling” of sounds, e.g., through vibrations. To this point, stimulator 222 may be configured to cause audio output device 236, or another one of external device(s) 238, to emit vibrations that are applied in a way so as to be felt by the subject (e.g., without arousing the subject). Vibrotactile stimulation may be particularly useful for elderly subjects, e.g., with reduced hearing capacity.

Notably, as alluded to above, stimulator 222 is configured to adaptively trigger auditory stimulation by modifying the “action” taken responsive to subsequent slow oscillation detections. In other words, stimulator 222 can adjust a time delay between detection and auditory stimulation to ensure that auditory stimulation is applied at precisely the right moment in the slow oscillation. It should be appreciated that characteristics of slow oscillations (e.g., the upstate) depend on the particularities of the subject, e.g., their age, pathologies, etc., as well as the sensors being used to measure brain wave activity. Thus, stimulator 222 is notable in its ability to adapt to any kind of slow oscillation, regardless of shape, frequency, etc. Additionally, it will be appreciated that synchronization of auditory stimulation with the slow oscillation itself is important in achieving desirable results. It is important to ensure that, when a stimulation is performed, it is synchronized with the brain wave activity signal that is being analyzed.

To this point, stimulator 222 may include a reinforcement learning model that modifies said time delay based on a reward value calculated by a reward calculator 224, as described below. In various implementations, the reinforcement learning model can be an n-bandit learning model, a multi-armed bandit learning model, a stochastic scheduling based model, or the like, where the timing of executing the “action” (e.g., the application of auditory stimulation) is optimized based on the assignment of a numeric value which is translated from a measurement of the effect of the action. In some implementations, the “actions” (e.g., time delay(s)) selected by stimulator 222 are based, at least in part, on characteristics of the subject, such as their age, gender, associated pathologies, and the like. Thus, the variations in slow oscillation characteristics between subjects are accounted for.

In some implementations, to select an initial action, stimulator 222 considers a temperature parameter (e.g., similar to Boltzmann decay) that is reduced exponentially (e.g., from 1 to 0). In some such implementations, stimulator 222 selects a random number (e.g., between 0 and 1) and, if the value is below the temperature parameter, subsequently selects a random “action”—in other words, a random time delay. In this regard, the “random action” is selected from a range of values that are adjusted according to general parameters for the subject, such as age, demographics, and so on. However, if the selected “random action” is not below the temperature parameter (e.g., which decreases as long as stimulations are being applied), stimulator 222 can select an “action value” from a Q-Table. As will be appreciated, a Q-table is a table that has numeric values associated to each one of a number of possible time delays or “actions” that stimulator 222 can select from. Thus, after stimulation is applied, reward calculator 224 (described below) can generate a reward value related to the stimulation and perform an iterative update on the Q-Table for “action” that was selected by stimulator 222 (e.g., which produced the reward).

Reward calculator 224 generally calculates the reward value (e.g., for use by stimulator 222 in reinforcement learning to adjust the time delay before stimulation) by parsing the components of a detected slow oscillation to determine an effect of the auditory stimulation on the slow oscillation. In some implementations, reward calculator 224 identifies a second “valley” in the signal, which is used to determine the reward for reinforcement learning; however, in other implementations, other or additional points in the signal could be considered. For example, the third and/or fourths valley in the signal could be included as different states (e.g., in which case the reinforcement learning scheme applied by stimulator 222 may be based on an iterated value function, such as Q-Learning). FIG. 5, which has been mentioned above, shows an example slow oscillation and identifies the points in the signal where localizer 212 detects the slow oscillation, zero crossing points, the second valley, etc.

In implementations where the reward is based on the second valley in the signal, in particular, reward calculator 224, identifies a first zero-crossing in the signal after detection (e.g., during the upstate) and a second zero-crossing after applying auditory stimulation (e.g., as the signal trends negative after the upstate). Based on these two zero-crossing points, reward calculator 224 identifies the second valley in the signal. Once the second valley in the signal is identified (e.g., as shown in FIG. 5), reward calculator 224 determines a raw value of the signal at the “second valley.” Then, a peak-to-peak amplitude between the second valley and the “second maximum” value of the signal (e.g., also shown in FIG. 5) is calculated as the “reward.” However, it should be appreciated that other techniques and/or parameters can be used as, or to calculate, a reward for reinforcement learning; thus, the present disclosure is not limiting in this regard. For example, in some implementations, reward calculator 224 may use an envelope function of the Hilbert transform of the signal between two frequencies (e.g., 0.5 and 4 Hz) to generate a reward value.

It should be appreciated that this “calibration” of stimulation timing (e.g., by applying stimulation, recording the effect, calculating a reward, adjusting timing for a subsequent stimulation, etc.) can be performed once during a “stimulation phase” of a treatment cycle (e.g., once per night) or at each different sleep stage. In this manner, timing can be adapted to the different types of slow oscillations and/or slow oscillation characteristics associated with different sleep stages. To this point, if stimulation results in arousal (e.g., as discussed below), it should be noted that the resulting brain wave activity may be discard or not used for reinforcement learning.

Memory 210 is further shown to include an arousal detector 226 and corresponding volume adjuster 228. Generally, arousal detector 226 is configured to detect that a subject is awake or, rather, has been aroused, e.g., due to auditory stimulation. Arousal detector 226 monitors the EMG signal provided by biometric sensor(s) 234 to calculate an average standard deviation of the EMG signal. Then, if the EMG signal exceeds three standard deviations of its average value, arousal detector 226 may output a signal indicating that the subject has potentially been aroused due to the auditory stimulation. Often, this is an indication that the volume of the auditory stimulation was too high. Accordingly, volume adjuster 228 may adjust the volume level of the auditory stimulation for subsequent applications.

Volume adjuster 228 may also be configured to determine an initial stimulation volume. In some implementations, for example, volume adjuster 228 can start at a predefined initial volume (or may randomly select an initial volume, e.g., from a range) and can incrementally increase the volume of each successive application of auditory stimulation until arousal is detected. For example, volume adjuster 228 may increase the volume of each pink noise burst by a set amount for each successively detected slow oscillation until arousal detector 226 detects arousal. Then, volume adjuster 228 may revert the volume back to the last volume level before arousal was detected. In other implementations, the initial (or final) stimulation volume is established during the calibration phase (e.g., in conjunction with generating the detection parameters, in some cases), as mentioned above. For example, stimulator 222 may be configured to emit a tone (e.g., a pink noise burst) or series of tones (e.g., three tones separated by a time interval) after a subject is determined to be in Stage 2 sleep. Then, if arousal detector 226 detects arousal, the volume is reduced by one step/interval; otherwise, the volume may be increased by one step/interval and the tone or series of tones repeated. In this manner, the stimulation phase is not interrupted by setting or adjusting the volume of stimulations.

It should be appreciated that CLAS system 200 can generally apply different stimulation protocols, e.g., regarding waiting time, type of slow wave component detection, stimulation timing, etc., based on the different subject demographics or pathologies. Thus, it should be understood that any of the parameters described herein (e.g., the detection parameters, timing or thresholds sleep stage detection, etc.) may be particularly calibrated to each individual subject, providing greater flexibility/adaptability across demographics and pathologies. In some cases, multiple different implementations of each component (or certain components) of memory 210 may be developed and/or trained for different demographics and pathologies. For example, a plurality of “stimulation profiles” may be generated, prior to using CLAS system 200 for treatment of a subject, based on information gathered from subjects of different demographics and/or having different pathologies to train multiple instances of CLAS system 200 or the memory components thereof. In some implementations, predefined stimulation profiles can be selected, e.g., when using CLAS system 200, based on the subject's age, gender, pathology, etc., to set initial parameters for the various components of memory 210. In this way, CLAS system 200 can easily be adapted for treatment of various demographics and pathologies. Further, additional calibration can be minimized (or the time to calibrate reduced) by implementing predefined stimulation profiles.

Still referring to FIG. 2, CLAS system 200 is also shown to include a communications interface 230 that facilitates communications with any external components or devices. For example, communications interface 230 can provide means for transmitting data to, or receiving data from, any of brain wave activity sensor(s) 232, biometric sensor(s) 234, audio output device 236, and/or external device(s) 238 (described below). In this regard, communications interface 230 can include any number and/or combination of wired and/or wireless communications interfaces (e.g., jacks, antennas, transmitters, receivers, transceivers, wire terminals, etc.) for conducting data communications. Further, communications via communications interface 230 can direct (e.g., local wired or wireless communications) and/or via a network (e.g., a WAN, the Internet, a cellular network, etc.). For example, communications interface 230 may include one or more Ethernet ports for communicably coupling CLAS system 200 a network (e.g., the Internet). In another example, communications interface 230 can include a wireless transceiver (e.g., a Wi-Fi transceiver, cellular or mobile phone communications transceivers, etc.) for communicating via a wireless communications network. In yet another example, communications interface 230 includes input/output jacks (e.g., sync boxes) suitable for receiving analog or digital signals from brain wave activity sensor(s) 232 and/or biometric sensor(s) 234, and/or transmitting signals to audio output device 236. In yet another example, communications interface 230 can include fiber optic transceivers for optical communications (e.g., to electrically isolate CLAS system 200 from input/line power).

External device(s) 238, as mentioned above, may include any number and/or type of computing device(s) that can interface with CLAS system 200, e.g., through communications interface 230. External device(s) 238 can include, for example, one or more mobile phones (e.g., smartphones), electronic tablets, laptops, desktop computers, workstations, servers, and other types of electronic devices. In some implementations, an external device (e.g., one of external device(s) 238) can include a user interface that allows a user to interact with CLAS system 200, e.g., remotely. For example, a smartphone may be remotely connected to CLAS system 200 for viewing data, adjusting parameters, or otherwise interacting with CLAS system 200. In this regard, external device(s) 238 may also include a stand-alone user interface that can display data from CLAS system 200 directly and/or that can receive user inputs for CLAS system 200. In another example, external device(s) 238 include a remote server (e.g., a cloud server) from which CLAS system 200 can obtain updates (e.g., software updates) or other data and/or to which CLAS system 200 can transmit data (e.g., for storage). In one such example, a remote server can be used to train any of the machine learning models described herein, e.g., to reduce computational requirements for CLAS system 200 (e.g., as training machine learning models can be computationally expensive), such that CLAS system 200 can receive the trained models for use.

Referring now to FIGS. 4A and 4B, two example diagrams that illustrate sensor placement on a subject—also referred to herein as a sensor “montage”—are shown, according to some implementations. FIG. 4A, in particular, shows example positioning of brain wave activity sensor(s) 232 and biometric sensor(s) 234 on and around the head/face of a subject. In this example, brain wave activity sensor(s) 232 are shown as F3, F4, C3, C4, P3, P4, which are generally positioned on the top of the subject's head. A ground electrode is also shown to be positioned on or around the subject's forehead. In this setup, six brain wave activity (e.g., EEG) channels can be recorded. In some implementations, one of the channels (e.g., C3) may be established as a default channel for slow oscillation detection; however, the default channel may be selectable. Biometric sensor(s) 234, which again include EMG and EOG sensors, are positioned at various points on the subject's face. The EMG sensors are labelled as EMG 1 and EMG 2, in this example. Likewise, the EOG sensors are labelled as EOG 1 and EOG 2, resulting in a bipolar channel for each of the EMG and EOG signals.

FIG. 4B shows another example sensor montage that includes just one of brain wave activity sensor(s) 232 and just one of biometric sensor(s) 234, shown as C3 and EMG2, respectively. Additionally, a ground electrode is shown, e.g., positioned on the subject's forehead. In some implementations, though not illustrated in FIG. 4A or FIG. 4B, all of the sensors/electrodes of brain wave activity sensor(s) 232 and biometric sensor(s) 234 are integrated into a substrate or headset that can be worn by the subject. In other implementations, each of brain wave activity sensor(s) 232 and biometric sensor(s) 234 can be individually attached to the subject's skin. It should further be appreciated that the specific position, number, and type of sensors included in a sensor montage, or otherwise positioned on the subject for capturing the above-mentioned EEG, EOG, and/or EMG signals, is not intended to be limited to the specific arrangements shown. Rather, FIGS. 4A and 4B are intended only as representative examples of suitable sensor layouts.

### Subject-Specific Detection Parameters

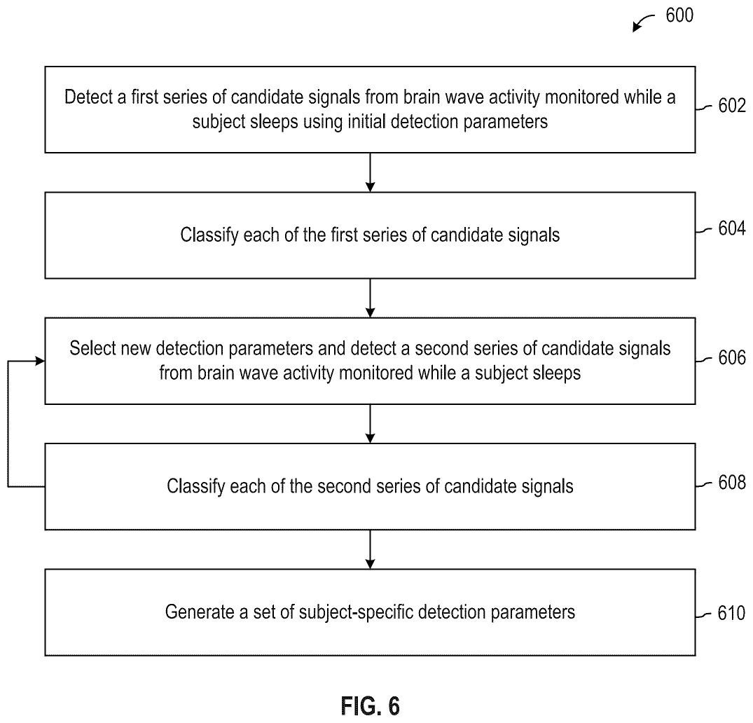

Referring now to FIG. 6, a flow chart of a process 600 for generating subject-specific slow oscillation detection parameters, according to some implementations. As mentioned above, subject-specific or “customized” slow oscillation detection is one notable feature of the disclosed auditory stimulation technique, allowing slow oscillations to be detected in a variety of different subjects with different demographics or pathologies. In some implementations, process 600 is implemented by CLAS system 200, as described above. In this regard, process 600 may be considered a “calibration” phase of CLAS system 200, which is performed prior to treatment/stimulation. It will be appreciated that certain steps of process 600 may be optional and, in some implementations, process 600 may be implemented using less than all of the steps. It will also be appreciated that the order of steps shown in FIG. 6 is not intended to be limiting.

At step 602, a first series of candidate signals is detected from brain wave activity monitored while a subject is asleep using an initial set of detection parameters. As discussed above, a “candidate signal” is a segment of a brain wave activity signal (e.g., an EEG signal) that is suspected of being associated with (e.g., the start of) a slow oscillation. Brain wave activity signals are obtained using suitable brain wave activity sensors, such as an electrode or electrode array for capturing EEG records, and are generally continuously recorded/measured, e.g., throughout process 600. With respect to CLAS system 200, EEG signals can be recorded by brain wave activity sensor(s) 232 and continuously analyzed by localizer 212 to detect candidate signals. To detect a candidate signal, successive segments (e.g., one second in length) of a brain wave activity signal are evaluated against initial detection parameters. The detection parameters, as mentioned above, can include one or more parameters that characterize a slow oscillation prior to a first peak (e.g., prior to hyperpolarization). In some implementation, the detection parameters include a peak-to-peak amplitude threshold and a negative threshold; however, more, fewer, or other parameters can be consider, as described above. When the initial detection parameters are met, a portion of the associated signal is recorded (e.g., time-locked) for further processing. The initial detection parameters may be randomly selected from a standardized set of values or may be predefined. In some implementations, initial detection parameters are selected based on a subject's demographics and/or known pathologies.

At step 604, each of the first series of candidate signals is classified. As mentioned above, classification can be performed using a unary classifier or, alternatively, a multi-class classifier. As such, each of the first series of candidate signals can be classified as either “indicative of a slow oscillation” or not, and/or each of the first series of candidate signals can be classified as a specific type of slow oscillation (e.g., type I, type II, false, etc.) or as having a specific slow oscillation characteristic. In any case, each of the first series of candidate signals is provided to a trained classifier (e.g., a machine learning model) which outputs a predicted class/label for each candidate signal portion and, optionally, a confidence score in the prediction. In this regard, the classifier provides a “ground-truth” for determining whether a candidate signal is, in fact, indicative of a slow oscillation and/or for determining a type of slow oscillation. In some implementations, each candidate signal detected at step 602 is evaluated with the classifier individually; thus, steps 602 and 604 of process 600 may repeat until the first “series” of candidate signals is developed. In other implementations, multiple candidate signals are detected and classified, e.g., in bulk.

At step 606, a new set of detection parameters is selected, and a second series of candidate signals is detected from brain wave activity monitored while the subject is asleep. In this regard, step 606 is generally substantially similar to step 602, where the new set of detection parameters is used to detect one or more candidate signals suspected of being associated with (e.g., the start of) a slow oscillation. The “new” set of detection parameters may be randomly selected from a standardized set of values or may be calculated from the initial set of detection parameters. In either case, the new set of detection parameters is generally different from initial set of detection parameters.

At step 608, each of the second series of candidate signals is classified. In this regard, step 608 may also be generally substantially similar to step 604, where each of the second series of candidate signals is provided to the trained classifier, which outputs a predicted class/label for each candidate signal portion and, optionally, a confidence score in the prediction. In some implementations, each candidate signal detected at step 606 is evaluated with the classifier individually; thus, steps 606 and 608 of process 600 may repeat until the second “series” of candidate signals is developed. In other implementations, multiple candidate signals are detected and classified, e.g., in bulk.

Additionally, as shown, steps 606 and 608 may be repeated any number of times to generate additional sets of candidate signals (e.g., a third set, a fourth set, etc.). With each repetition, a new set of detection parameters may be used such that each subsequently set of candidate signal is detected using different detection parameters. Ultimately, this repetition of steps 606 and 608 results in two or more sample datasets from both possible classes, e.g., as determined by the classifier; for example, a first dataset of signal segments (e.g., candidate signals) that are determined to be associated with slow oscillations and a second dataset of signal segments that are determined to be false positives or not associated with slow oscillations, or multiple datasets of signal segments associated with different types/classes of slow oscillations.

At step 610, a set of subject-specific detection parameters is generated based on the sample datasets. In some implementations, the subject-specific detection parameters are generated adjusting initial parameter values. For example, the initial peak-to-peak amplitude threshold and negative threshold—or a predefined peak-to-peak amplitude threshold and negative threshold that is not necessarily previously used in process 600—can be increased or decreased. In other implementations, the subject-specific detection parameters are generated by adjusting one of a standardized set of detection parameters (e.g., used to randomly select the initial detection parameters as mentioned above) or a last-used set of detection parameters. In any case, the detection parameters may be adjusted using a linear thresholding algorithm, such as a linear perceptron, SVM, Otsu mode, or the like. As mentioned above, the linear thresholding algorithm is used to find the separation hyperplane (e.g., in two dimensions for two free parameters) for estimating parameters.

### Detection and Stimulation

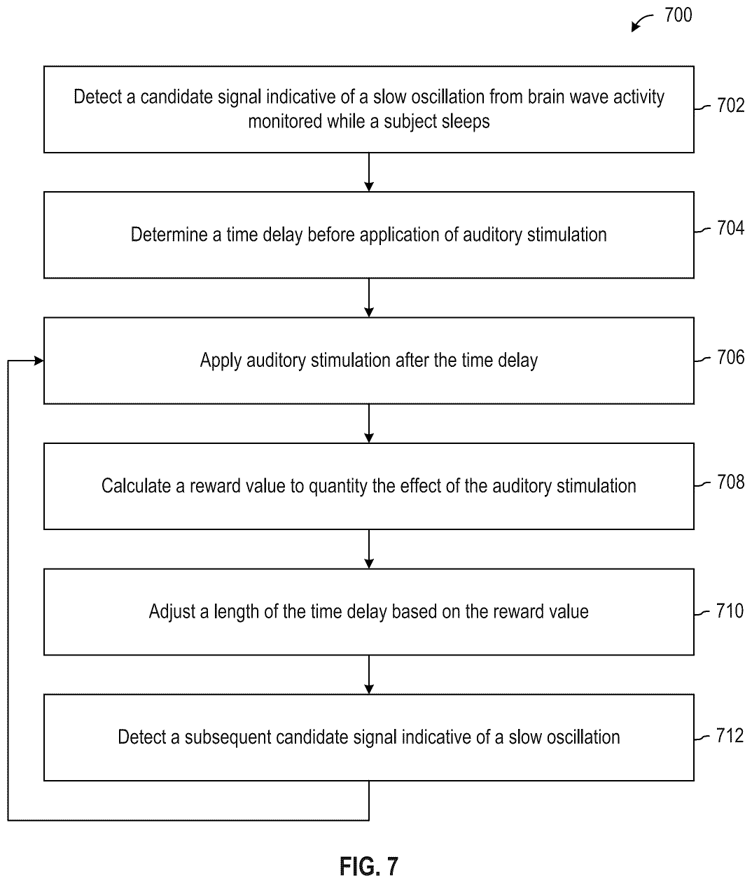

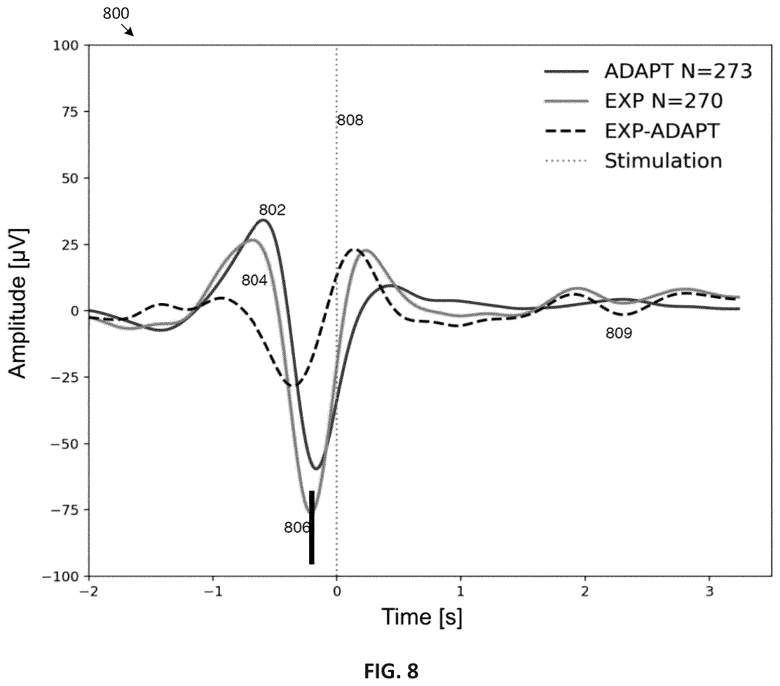

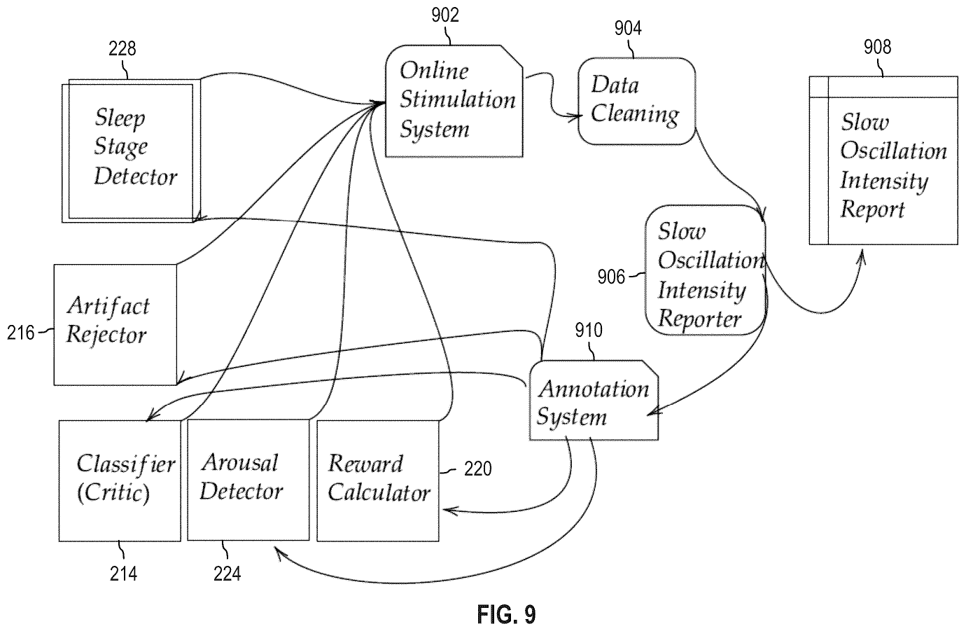

Referring now to FIG. 7, a flow chart of a process 700 for adaptively detecting slow oscillations and applying auditory stimulation, according to some implementations. As mentioned above, adaptive stimulation is another notable feature of the disclosed auditory stimulation technique, helping to ensure that stimulation is applied at precisely the right moment in a slow oscillation despite the unpredictable and varying nature of slow oscillations, e.g., among subjects of different demographics or with different pathologies. In some implementations, process 700 is implemented by CLAS system 200, as described above. In this regard, process 700 may be considered a “stimulation” or “treatment” phase of CLAS system 200, which is performed after the calibration described above with respect to process 700. It will be appreciated that certain steps of process 700 may be optional and, in some implementations, process 700 may be implemented using less than all of the steps. It will also be appreciated that the order of steps shown in FIG. 7 is not intended to be limiting.

At step 702, a candidate signal indicative of a slow oscillation is detected from brain wave activity monitored while the subject is asleep. As discussed above, the “candidate signal” is a segment of a brain wave activity signal (e.g., an EEG signal) that is suspected of being associated with (e.g., the start of) a slow oscillation. Brain wave activity signals are obtained using suitable brain wave activity sensors, such as an electrode or electrode array for capturing EEG records, and are generally continuously recorded/measured, e.g., throughout process 700. With respect to CLAS system 200, EEG signals can be recorded by brain wave activity sensor(s) 232 and continuously analyzed by localizer 212 to detect candidate signals. To detect a candidate signal, successive segments (e.g., one second in length) of a brain wave activity signal are evaluated against subject-specific detection parameters. The subject-specific detection parameters can include, for example, a peak-to-peak amplitude threshold and a negative threshold, and are generated using process 600 as discussed above. When the subject-specific detection parameters are met, a portion of the associated signal is recorded (e.g., time-locked) for further processing.

In some implementations, as part of detecting the candidate signal, a potential candidate signal is evaluated to determine whether the signal is, in fact, a suitable candidate signal or whether the signal is an artifact (e.g., noise). In some cases, signals that are determined to be an artifact (e.g., by artifact rejector 220) are not suitable for further processing and are rejected. Artifact rejector 220, for example, compares the brain wave activity signals received from brain wave activity sensor(s) 232 to a predefined threshold and, if the threshold condition is met, rejects the corresponding signal components as an artifact. In conjunction, process 700 may be suspended until a refractory period (e.g., one minute) has passed.

At step 704, a time delay before application of auditory stimulation is determined. As mentioned above, the time delay is an “action” selected by stimulator 222 and is generally defined as a number of time steps between detection of the candidate signal and/or detection of a stimulation trigger point (e.g., a negative-to-positive shift in the value of the signal) and application of auditory stimulation. To this point, the brain wave activity signal may be continuously analyzed (e.g., at each of a plurality of successive time steps) to identify when the stimulation trigger point, as discussed above. When the detected stimulation trigger point is detected, a time delay (e.g., “action”) is selected, and a corresponding timer or counter is subsequently initiated. In some implementations, the time delay may be selected from a table and/or initially, a default time delay may be selected. Additional details are provided above with respect to FIG. 2.

At step 706, the auditory stimulation is applied after the time delay. In other words, auditory stimulation is triggered once the aforementioned timer or counter expires or reaches a set time/count. In some implementations, applying auditory stimulation includes emitting sound via an audio output device (e.g., audio output device 236) and/or causing an audio output device to output a sound. As mentioned above, auditory stimulation is typically a pink noise burst of a set length (e.g., 200 ms). Accordingly, the audio output device can include any device that is capable of producing and emitting pink noise as prescribed. For example, the audio output device may be a headset (e.g., a pair of headphones) or a speaker positioned next to, or near, the subject. To this point, in some implementations, the audio output device may be configured to be worn by the subject when they are asleep. In some implementations, auditory stimulation includes two pink noise bursts—a first pink noise burst emitted after the time delay and a second pink noise burst emitted after a refractory period, e.g., of one minute. In some implementations, vibrotactile stimulation can also be applied (e.g., in conjunction with the auditory stimulation), as mentioned above.

At step 708, a reward value is calculated to quantify the effect of the auditory stimulation. The reward value is generally calculated by parsing the components of a detected slow oscillation—or, more specifically, by parsing the brain wave activity signal before and after application of the auditory stimulation—to identify an effect of the applied simulation. In some implementations, the reward value is based on a second “valley” in the signal (e.g., see FIG. 5). In some such implementation, parsing the brain wave activity includes identifies a first zero-crossing in the signal after detection (e.g., during the upstate) and a second zero-crossing after applying auditory stimulation (e.g., as the signal trends negative after the upstate). Based on these two zero-crossing points, the second valley can be identified. However, as discussed above, the reward value can alternatively be calculated from other characteristics of the resulting brain wave activity signal.

At step 710, a length of the time delay is adjusted based on the reward value. To this point, as mentioned above, the timing of auditory stimulation is adaptable to account for variations in slow oscillation characteristics, helping to ensure that stimulation is applied at the ideal moment in the slow oscillation waveform. In some implementations, a reinforcement learning technique is applied to adjust the time delay. In various implementations, the reinforcement learning technique is based on an n-bandit learning, a multi-armed bandit learning, a stochastic scheduling, or the like, where the timing of executing the “action” (e.g., the application of auditory stimulation) is optimized based on the assignment of a numeric value which is translated from a measurement of the effect of the action.

At step 712, another candidate signal indicative of a subsequent slow oscillation is detected and process 700 returns to step 706. In other words, steps 706-712 of process 700 can repeat such that subsequent slow oscillations are detected, auditory stimulation is applied after a time delay, a reward value is calculated, and the time delay is adjusted based on the reward value. In this manner, the time delay is iteratively adjusted so that each subsequent auditory stimulation is applied at as close to an “optimal” point in a slow oscillation as possible. It should be therefore appreciated that steps 706-712 of process 700 can repeat a set number of times or until the difference between successive time delays is below a threshold amount. Alternatively, steps 706-712 of process 700 can be repeated indefinitely (e.g., throughout a treatment session, overnight, etc.) to continuously adjust the time delay between detection of slow oscillations and application of auditory stimulation. It should be noted, as discussed above, that this “calibration” of stimulation timing (e.g., based on the repetition of steps 706-712 of process 700) can be performed once or at each different sleep stage.

### Experimental Results

Referring now to FIG. 8, a graph 800 of an example slow oscillation with and without an applied auditory stimulus is shown, according to some implementations. In particular, graph 800 illustrates the effects of processes 600 and 700 and, more broadly, the use of CLAS system 200, as described above. Graph 800 includes two lines—a first line 802 illustrating the EEG signal of a slow oscillation without auditory stimulation and a second line 804 illustrating the EEG signal of a slow oscillation with auditory stimulation. Lines 802 and 804 were generated, in this example, from stimulus-locked event related potential curves of 270 and 273 signal segments for each condition. A detection line 806 indicates the point at the slow oscillations were detected, e.g., by localizer 212. A stimulation area 808 indicates a time period when a pink noise burst (e.g., auditory stimulation) was applied, e.g., with respect to line 804. Line 809 shows the difference between 802 and 804. In this regard, line 804 therefore clearly illustrates the effects of properly timed auditory stimulation, e.g., using CLAS system 200, by enhancing the shape of the slow wave and producing a “second valley.”

### Model Training

Referring now to FIG. 9, a block diagram of the interaction between components of the system of FIG. 2 for developing a training data set, according to some implementations. Specifically, the diagram in FIG. 9 illustrates a semi-supervised process for generating annotated training data, e.g., for training classifier 214 as discussed above. Initially, a training dataset of brain wave activity records, e.g., collected from sleeping subjects using CLAS system 200, is obtained. This process may incorporate, in some aspects, “human-in-the-loop” annotation. In the example of FIG. 9, an online stimulation system 902 represents a portion of CLAS system 200 used for collecting said brain wave activity signals to generate a training data set. In some implementations, a data cleaner 904 cleans and/or organizes the collected brain wave activity data, e.g., by removing outliers, noise, etc. A slow oscillation intensity reporter 906 the takes the training dataset of brain wave activity data and generates a summary—shown as slow oscillation intensity report 908—that details information regarding a location of each slow oscillation as it was detected (e.g., by localizer 212). In some implementations, slow oscillation intensity reporter 902 also marks the morphological parameters of each slow oscillation, e.g., as shown in FIG. 5. The information from stimulations can also be registered. Experts can then interact with an annotation system 910 to identify and/or mark each slow oscillation independently (e.g., marking from the first max to the upstate). This information is then used to generate the training dataset used by classifier 214 to provide the ground truth.

In some implementations, in addition to, or in lieu of, manual annotation, the training dataset is provided to a machine learning model for preliminary labeling. The machine learning model may, in particular, be designed to apply labels to the brain wave activity records as noted below. For example, in some such implementations, the machine learning model may include a classifier for identifying (e.g., classifying) slow oscillations in the records and applying labels. Whether manually, automatically, or through a combination thereof, the brain wave activity records may be annotated with any of the following labels: (i) true positives, which are slow oscillations that were correctly identified by localizer 212; (ii) false positives, which are slow oscillations that were incorrectly identified by localizer 212; and (iii) false negatives, which are slow oscillations that were missed by localizer 212. False positives, for example, are often very low amplitude oscillations that didn't meet the criteria to be considered slow oscillations or are artifacts that were not detected by sleep stage detector 216. False negatives are, in general, slow oscillations that didn't meet the detection parameters utilized by localizer 212 for one reason or another and were therefore neglected. Additionally, some signal components may be labeled as “true negatives,” which are often “EEG ground swells” and cannot really be identified properly by localizer 212. As mentioned above, the annotated brain wave activity records can then be used to train classifier 214, e.g., to accurately identify slow oscillations.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

As used herein, Slow Oscillations (SO) are oscillations or transient signal components that appear during SWS and are characterized as 0.5-4 Hz. Sometimes they are also referred as Delta Waves.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A method of auditory stimulation to affect sleep of a subject, the method comprising:
monitoring a brain wave activity signal while the subject is asleep;
detecting, from the brain wave activity signal, an indication of the start of a slow oscillation using a set of detection parameters generated for the subject, the set of detection parameters based on characteristics of the slow oscillation prior to hyperpolarization;
determining a time delay to apply before auditory stimulation responsive to detecting the start of the slow oscillation, wherein the time delay is applied after detecting a trigger point in an upstate of the slow oscillation or after depolarization;
applying auditory stimulation to affect the slow oscillation after the time delay, the auditory stimulation emitted by an audio output device; calculating a reward value for the auditory stimulation by evaluating the brain wave activity signal after applying the auditory stimulation, the reward value calculated in part by parsing the brain wave activity signal; and
adjusting, based on the reward value, a length of the time delay to be applied prior to subsequent applications of the auditory stimulation associated with subsequent slow oscillations.

2. The method of claim 1, further comprising generating the set of detection parameters for the subject prior to detecting the start of the slow oscillation by:
generating a series of candidate signals by iteratively detecting, from the brain wave activity signal, a signals using a preliminary set of detection parameters, wherein the preliminary set of detection parameters are adjusted between iterations;
determining a class for each of the series of candidate signals using a classifier; and
generating the set of detection parameters for the subject based on the class of each of the series of candidate signals.

3. The method of claim 2, wherein the set of detection parameters are for the subject generated by using a thresholding algorithm to find a separation hyperplane between detection parameters.

4. The method of claim 1, wherein the brain wave activity signal comprises an electroencephalogram (EEG) signal.

5. The method of claim 1, wherein a refractory period is applied between detection of the start of the slow oscillation and detection of the subsequent slow oscillations.

6. The method of claim 1, further comprising: determining, after detecting the indication of the start of the slow oscillation but prior to application of the auditory stimulation, whether the brain wave activity signal exceeds a threshold amplitude; and rejecting the indication of the start of the slow oscillation as an artifact if the brain wave activity signal exceeds the threshold amplitude.

7. The method of claim 1, wherein the reward value is calculated by: parsing the brain wave activity signal to identify: (i) a first zero crossing after detection of a downward deflection of the slow oscillation, (ii) a second zero crossing after application of the auditory stimulation, (iii) the second valley in the brain wave activity signal, wherein the second valley is between the first zero crossing and the second zero crossing, and (iv); and a peak after the second valley; and measuring a peak-to-peak value of the brain wave activity signal between the second valley and the peak.

8. The method of claim 1, wherein the indication of the start of the slow oscillation is detected by continuously analyzing one second segments of the brain wave activity signal.

9. The method of claim 1, wherein the audio output device configured to be worn by the subject or positioned within a set distance of the subject while the subject is asleep.

10. The method of claim 1, further comprising setting a volume of the auditory stimulation by: monitoring biometric signals associated with the subject in conjunction with the brain wave activity signal; and incrementally increasing the volume of the auditory stimulation until arousal of the subject is detected based on the biometric signals, wherein the volume of the auditory stimulation is set at one step/interval below a point where arousal is detected.

11. The method of claim 10, wherein the biometric signals comprise at least one of electrooculography (EOG) signals or electromyography (EMG) signals.

12. A system for auditory stimulation to affect sleep, the system comprising:
a first sensor configured to record brain wave activity in a subject; an audio output device;
one or more processors; and
memory having instructions stored thereon that, when executed by the one or more processors, cause the system to:
monitor brain wave activity signals while the subject is asleep using the first sensor; detect, from the brain wave activity signal, an indication of the start of a slow oscillation using a set of detection parameters generated for the subject, the set of detection parameters based on characteristics of the slow oscillation prior to hyperpolarization;
determine a time delay to apply before auditory stimulation responsive to detecting the start of the slow oscillation, wherein the time delay is applied after detecting a trigger point in an upstate of the slow oscillation or after depolarization;
emitting, by the audio output device, auditory stimulation to affect the slow oscillation after the time delay;
calculate a reward value for the auditory stimulation by evaluating the brain wave activity signal after applying the auditory stimulation, the reward value calculated in part by parsing the brain wave activity signal; and
adjust, based on the reward value, a length of the time delay to be applied prior to subsequent applications of the auditory stimulation associated with subsequent slow oscillations.

13. The system of claim 12, wherein the instructions further cause the system to:
generate the set of detection parameters for the subject prior to detecting the start of the slow oscillation, including to:
generate a series of candidate signals by iteratively detecting, from the brain wave activity signal, a signals using a preliminary set of detection parameters, wherein the preliminary set of detection parameters are adjusted between iterations;
determine a class for each of the series of candidate signals using a classifier; and
generate the set of detection parameters for the subject based on the class of each of the series of candidate signals.

14. The system of claim 13, wherein the set of detection parameters are for the subject generated by using a thresholding algorithm to find a separation hyperplane between detection parameters.

15. The system claim 12, wherein the first sensor is configured to record electroencephalogram (EEG) signals.

16. The system of claim 12, wherein a refractory period is applied between detection of the start of the slow oscillation and detection of the subsequent slow oscillations.

17. The system of claim 12, wherein the instructions further cause the system to: determine, after detecting the indication of the start of the slow oscillation but prior to application of the auditory stimulation, whether the brain wave activity signal exceeds a threshold amplitude; and reject the indication of the start of the slow oscillation as an artifact if the brain wave activity signal exceeds the threshold amplitude.

18. The system of claim 12, wherein calculating the reward value includes to: parse the brain wave activity signal to identify: (i) a first zero crossing after detection of a downward deflection of the slow oscillation, (ii) a second zero crossing after application of the auditory stimulation, (iii) the second valley in the brain wave activity signal, wherein the second valley is between the first zero crossing and the second zero crossing, and (iv); and a peak after the second valley; and measure a peak-to-peak value of the brain wave activity signal between the second valley and the peak.

19. The system of claim 12, wherein the indication of the start of the slow oscillation is detected by continuously analyzing one second segments of the brain wave activity signal.

20. The system of claim 12, wherein the audio output device configured to be worn by the subject or positioned within a set distance of the subject while the subject is asleep.

21. The system of claim 12, further comprising a second sensor for recording biometric signals associated with the subject, wherein the instructions further cause the system to: monitor the biometric signals in conjunction with the brain wave activity signals; and incrementally increase the volume of the auditory stimulation until arousal of the subject is detected based on the biometric signals, wherein the volume of the auditory stimulation is set at one step/interval below a point where arousal is detected.

22. The system of claim 21, wherein the biometric signals comprise at least one of electrooculography (EOG) signals or electromyography (EMG) signals.

23. A non-transitory computer readable medium having instructions stored thereon that, when executed by one or more processors, cause a device to:
monitor brain wave activity signals while a subject is asleep;
detect, from the brain wave activity signal, an indication of the start of a slow oscillation using a set of detection parameters generated for the subject, the set of detection parameters based on characteristics of the slow oscillation prior to hyperpolarization;
determine a time delay to apply before auditory stimulation responsive to detecting the start of the slow oscillation, wherein the time delay is applied after detecting a trigger point in an upstate of the slow oscillation or after depolarization;
apply auditory stimulation to affect the slow oscillation after the time delay; calculate a reward value for the auditory stimulation by evaluating the brain wave activity signal after applying the auditory stimulation, the reward value calculated in part by parsing the brain wave activity signal; and
adjust, based on the reward value, a length of the time delay to be applied prior to subsequent applications of the auditory stimulation associated with subsequent slow oscillations.

24. The computer readable medium of claim 23, wherein the instructions further cause the device to: generate the set of detection parameters for the subject prior to detecting the start of the slow oscillation, including to: generate a series of candidate signals by iteratively detecting, from the brain wave activity signal, a signals using a preliminary set of detection parameters, wherein the preliminary set of detection parameters are adjusted between iterations; determine a class for each of the series of candidate signals using a classifier; and generate the set of detection parameters for the subject based on the class of each of the series of candidate signals.

25. The computer readable medium of claim 23, wherein calculating the reward value includes to: parse the brain wave activity signal to identify: (i) a first zero crossing after detection of a downward deflection of the slow oscillation, (ii) a second zero crossing after application of the auditory stimulation, (iii) the second valley in the brain wave activity signal, wherein the second valley is between the first zero crossing and the second zero crossing, and (iv); and a peak after the second valley; and measure a peak-to-peak value of the brain wave activity signal between the second valley and the peak.
